(19) United States
(12) Patent Application Publication
(10) Pub. No.: US 2003/0108893 A1
(43) Pub. Date: Jun. 12, 2003

(54) Method for establishing neurogically relevant properties of a material

(76) Inventors: Claudia Rudolph, Munchen (DE); Gerhard Weidner, Munchen (DE); Marius Hoener, Basel (CH); Karlheinz Tovar, Bad Homburg (DE); Peter Riederer, Wurzburg (DE); Manfred Gerlach, Hammelburg (DE); Ralf Baumeister, Grobenzell (DE)

Correspondence Address: Finnegan Henderson Farabow, Garrett & Dunner, Franklin Square Building, 1300 I Street NW Suite 700, Washington, DC, 20005-3315, US

(21) Appl. No.: 10/168,558

(22) PCT Filed: Dec. 20, 2000
(86) PCT No.: PCT/EP00/13022

(30) Foreign Application Priority Data
Dec. 22, 1999 (DE) 1996226.9

(51) Int. Cl. C12Q 1/68; A61K 49/00

(52) U.S. Cl. 435/6; 424/9.2

(57) ABSTRACT

The invention relates to a method for establishing the neurologically relevant properties of a material, for which a nematode is used.

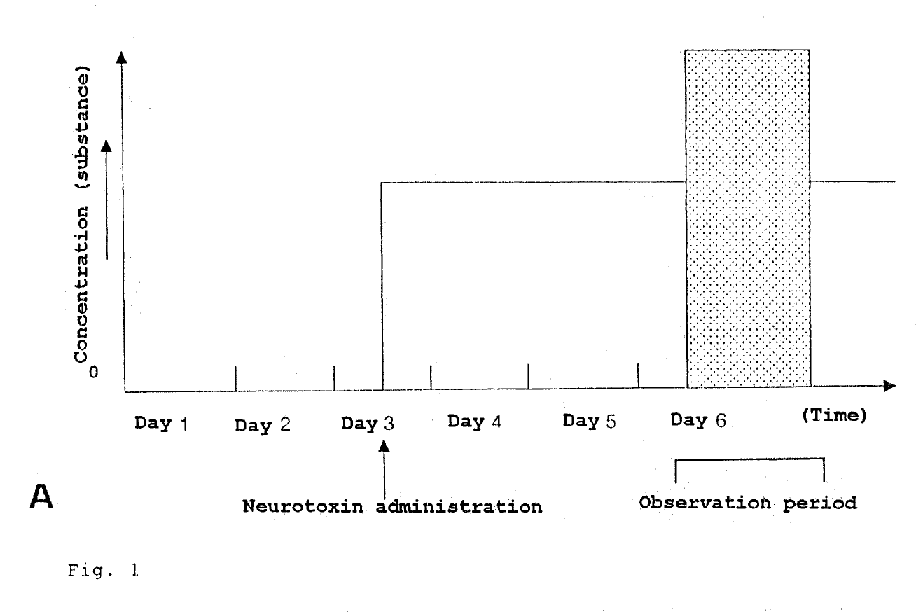

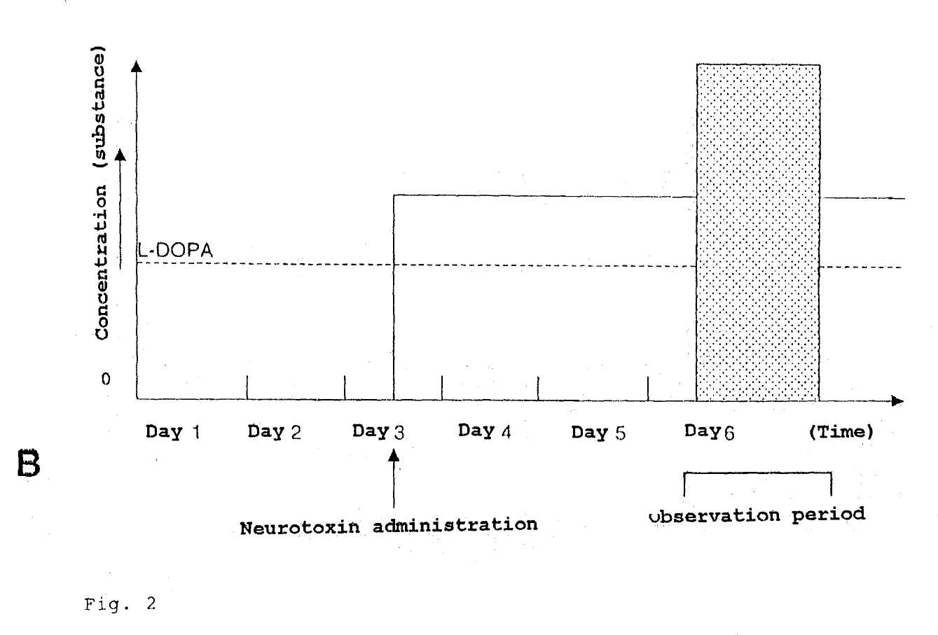

The present invention relates to a method for establishing neurological properties of a material; in particular, the present invention relates to a method for investigating the suitability of a material as a drug for mammals, that is for humans, inter alia.

On average, it takes a total of from 10 to 12 years to develop new drugs. Most of this time is taken up in initially using a selective approach to find a material which is likely to be suitable for a particular indication. Frequently, the number of substances which are taken into consideration initially runs into hundreds of thousands. Consequently, there is a particular need for a simple and rapid test method which can be used for categorizing the substances to be investigated as being in principle suitable or unsuitable. However, at present, there are no known methods which can be used for making predictions, with a high degree of reliability, concerning any possible efficacy in humans (or other mammals). While it is of course true that animal models are available for this purpose, they are either not particularly reliable as regards their predictive value and/or are very elaborate. Furthermore, conventional animal models are to an increasing extent being rejected generally since they cause suffering to the animals (for example dogs and monkeys). On the other hand, it is regularly demanded that animal models should bear a great resemblance to the human body so as to ensure a certain degree of probability that the knowledge gained from the animal experiments can be applied to humans.

There is a particular need for an animal model which has a high predictive value and can either replace conventional animal models or at least reduce the number of experiments carried out with higher organisms (such as mammals). This need exists, in particular, in connection with a search for remedies which are able to influence diseases of the human central nervous system, such as Parkinson's disease.

WO99/02652 relates to transgenic nematodes (threadworms) which can be used as model systems for triplet-repeat neurological diseases.

The present invention is now based on the finding that, when active compounds which have a neurotoxic effect in humans, such as 6-hydroxy-dopamine (6-OHDA) or 1-methyl-4-phenyl-1,2,3,6-tetrahydropyridine (MPTP), or its derivatives or chemical precursors, is/are administered to nematodes (threadworms), the latter then exhibit behavioral anomalies or changes in their morphology. The present invention is based on the insight that, on the one hand, the administration of neurotoxic substances to threadworms results in an anomaly in morphology or behavior and that, on the other hand, the anomaly which has thereby been brought about can be used as a basis for investigating the effect of a particular material on the threadworm nervous system which has been damaged in this way. As an accompaniment to this, the present invention can also be used for making a prediction with regard to the effect of this material, which has been identified as having an action in the threadworm, on disease-associated changes in the human central nervous system (and also that of other mammals).

The invention consequently relates to a method for establishing neurologically relevant properties of a material, with use being made of at least one nematode to which at least one neurologically relevant material (agonist or antagonist) and, where appropriate, a further neurologically relevant material having an opposite effect (antagonist, agonist) is/are administered.

This method also comprises establishing a variation in, or retention or restoration of, a normal behavior or a normal state. The observations which are made in this connection make it possible to draw a qualitative conclusion about the neurologically relevant properties of the first and/or second neurologically relevant material. In connection with the present invention, “neurologically relevant” means that the material can influence the central nervous system of humans, in particular, for example as a neurotoxic substance or as a substance or material which can abolish or alleviate the effect of the neurotoxic substance. Within the meaning of the present invention, the term “material” signifies that either a single substance or compositions consisting of several substances is/are used.

The invention consequently relates to a method for establishing a neurologically relevant property of a material, with use being made of at least one nematode to which at least one neurologically relevant material (agonist/antagonist) and, if appropriate, a second neurologically relevant material acting in the opposite direction (antagonist/agonist) is/are administered, and with a variation in, or, when a first and second neurologically relevant material are used, a retention or restoration of, a normal state enabling an observation to be made with regard to the neurological relevance of at least one of the materials employed.

The method according to the invention can be used, in particular, for establishing whether a particular material in all probability exerts a positive influence on disease-associated changes in the central nervous system of humans, in particular. In this case, the method according to the invention will regularly make use of a neurotoxic substance as the first neurologically relevant material. A nematode which, prior to, at the same as or after being exposed to a neurotoxic substance, such as 6-OHDA or MPTP, is exposed to a second neurologically relevant material, whose neurological relevance has still to be established, exhibits a change which enables unambiguous conclusions to be drawn with regard to the neurological relevance under investigation. Thus, as a consequence of the influence of a neurotoxicological substance, the nematode exhibits an anomaly in its morphology or its behavior which, depending on the second neurologically relevant material employed, can either be entirely or partially reversed or else is not even expressed at all.

In the simplest case, the method according to the invention can be used for establishing the neurologically relevant properties, for example neurotoxic properties, of a material which has not yet been investigated in this regard. In this case, only a single neurologically relevant substance would be administered to the nematode. However, the opportunity presents itself of additionally obtaining a confirmation of the establishment of a neurotoxic property by in turn employing, as the second neurologically relevant material, a material possessing an opposing neurologically relevant property. If the induction of the previously described anomaly in a nematode is abolished by the second neurologically relevant substance which acts in the opposite direction, this then only confirms the previously established neurotoxic property.

Expressed in another way, the invention can also be regarded as being a method for investigating the suitability of a material as a drug, with use being made of at least one nematode to which at least one neurotoxic material, on the one hand, and the material to be investigated, such as L-3,4-dihydroxyphenylalanine (L-DOPA), on the other hand, are administered.

As a rule, it will be sufficient, for establishing a suitability or non-suitability, to qualitatively record the divergence from a previously observed anomaly. Since the anomaly which is induced is very pronounced and significant (see details below), it will be easy to observe a divergence. Relatively large populations of nematodes will be routinely investigated and a qualitative divergence can essentially be expressed as a percentage, namely as a percentage of the animals which no longer exhibit a particular behavior or morphology which has been elicited by the effect of a neurotoxic substance, and which is changed as compared with the normal state (such as a change in neuronal structure), after they have been exposed to the material.

In analogy with the above-described invention, this method is also suitable for identifying genes which are involved in expressing the effect of neurotoxic substances. The invention consequently consists of a method in which nematodes are initially subjected to a mutagenesis and a neurologically relevant material, as described above, is then administered to the animals which have been genetically altered in this way, or to their descendants. The animals in which it is not possible to observe, any anomalies in behavior or morphology, or in which the anomalies in behavior or morphology are different from those in nonmutagenized animals, can carry genes which, have been altered, inactivated or destroyed by the mutagenesis and whose products have no action or act in another way than in nonmutagenized animals. The described method is consequently used for identifying genes which mediate or suppress the expression of the effect of neurotoxic substances, for example.

The invention consequently furthermore relates to a method for identifying neurologically relevant genes in which nematodes are initially deliberately subjected to a mutagenesis and, the genetically altered animals, or their descendants, are, after that, exposed to at least one neurologically relevant material, as previously described. In connection with the present aspect of the invention, “neurologically relevant gene” means that the gene is in some _way involved in the expression of the effect of a neurologically relevant material. This neurological relevance can lie, for example, in the fact that the mutated nematode displays a reaction (behavior or morphology) toward neurotoxic substances which is different from that of the nonmutated nematode. However, the neurological relevance of a gene can also manifest itself in the fact that, following exposure to a neurotoxic material, there then appears an anomaly which cannot be reversed or suppressed by exposing the mutated nematodes to a neurologically relevant material which has an opposite effect.

The response to the administration of neurotoxic substances which act on the central nervous system was investigated in nematodes of the genus Caenorhabditis and, in particular, in Caenorhabditis elegans. In this threadworm, a characteristic phenotype, which is expressed, for example, in movement disturbances, a reduced number of progeny or morphological changes,; is observed both in the larval stages L1, L2, L3 and L4 (see “The Nematode Caenorhabditis elegans”; Wood et al., Cold Spring Harbor Laboratory, 1988) and in the adult stage following incubation with a neurotoxic substance. This can be ascertained by observing the phenotype microscopically, by determining the number of progeny per animal and time period or by detecting nerve cells which have been labeled with particular dyes (e.g. GFP fusion proteins) and whose color changes after they have been acted upon by a neurotoxic substance. These neurotoxic substances evidently interfere in impulse transmission or, in a general manner, in the function or synaptic linkage of the nerves of the threadworm in such a way that this leads to uncontrolled movement sequences which are comparable with the Parkinson symptoms which are seen in humans.

It has now been found that certain chemical substances which exhibit pharmacological effects in humans also partially or completely reversed or prevented either behavioral anomalies or morphological changes in nematodes, despite the latter having been treated with a neurotoxic substance.

It is possible to expose the nematodes to the materials to be investigated in a wide variety of ways. For example, it is possible to culture the animals in Petri dishes containing an agar which contains neurotoxic substances, on the one hand, and the substances of interest on the other. However, it is also possible to bathe the animals, in a simple manner, in the materials to be investigated. As well as being particularly simple, this variant also makes it possible to standardize the concentrations more easily and to achieve a higher throughput in a screening system. For an investigation, use is made of populations of the animals, which populations are then incubated with different solutions of the materials and at different concentrations. The inventors' experiments have now demonstrated that substances which are able to reverse neurotoxically induced Parkinson in both humans and non-human primates are also able to do this in C. elegans. This shows that C. elegans can be used as a suitable model system for Parkinson. The observation, namely the reversion or prevention of the behavioral anomaly or the morphological change following neurotoxin treatment, makes it possible to draw a conclusion with regard to the possible activity and site of action of the material whose suitability as a drug is under investigation.

The inventors' investigations have consequently demonstrated that the use of C. elegans as an animal model for investigating materials with regard to their suitability as drugs in humans or other mammals enables reliable predictions to be made, in particular as far as the reactions to neurotoxic substances, and the reversion or prevention of these reactions, are concerned. It has been found that it is readily possible to bring about the behavioral anomalies by adding neurotoxic substances to nematodes kept in liquid culture. A “normal” behavioral pattern (=wild-type behavior) can be described by:

I) the bodies of the animals moving in a wavy manner in their longitudinal direction;

II) the nematodes making beating movements in all the developmental stages of the animals.

It was observed that, when other substances were not added, regularly at least 80% of the animals normally exhibited a behavior as described in I) and/or II).

The invention is explained in the following examples.

### EXAMPLES

Per assay, about 100 threadworm (C. elegans) test animals at a defined stage of development (e.g. L1 larval stage) were incubated in liquid medium at constant temperature (e.g. 20° C.). In order to investigate the behavior or the morphology of the animals in more detail, they were exposed, for from a few hours to several days, to the neurotoxic substance MPTP at concentrations of between 16 and 500 μg/ml (e.g. after three days in Figure A).

In an observation period lasting from a few minutes to several days (on day 6 in Figures A and B), a characteristic phenotype was observed. This can be expressed in severe movement disturbances, a reduced number of progeny or morphological changes.

100 threadworm test animals were once more used, as previously described, in a second experiment. These animals were inter alia preincubated for three days with L-DOPA and the neurotoxic substance MPTP was once again added on the third day.

Astonishingly, after an incubation (from a few hours to several days; e.g. after three days of preincubation in Figure B) with L-DOPA, the phenotype which is brought about by MPTP is not expressed.

This consequently proves that substances which reverse Parkinson symptoms in other animal models also do this in C. elegans.

1. A method for establishing a neurologically relevant property of a material, with use being made of at least one nematode to which at least one neurologically relevant material (agonist/antagonist) and, where appropriate, a second neurologically relevant material which acts in the opposite direction (antagonist/agonist) is/are administered, and with a variation in, or, when a first and second neurologically relevant material are used, a retention or restoration of, a normal state enabling an observation to be made with regard to the neurological relevance of at least one of the materials employed.

2. The method as claimed in claim 1, characterized in that the nematode belongs to the phylum Nematoda.

3. The method as claimed in claim 2, characterized in that the nematode belongs to the genus Caenorhabditis.

4. The method as claimed in any one of the preceding claims, characterized in that the first neurologically relevant material comprises a neurotoxic substance.

5. The method as claimed in claim 4, characterized in that the second neurologically relevant material is investigated for its suitability as a material for treating diseases of the central nervous system.

6. The method as claimed in claim 4 or 5, characterized in that the neurotoxic substance is 6-hydroxydopamine or 1-methyl-4-phenyl-1,2,3,6-tetrahydropyridine, or a precursor or a derivative of the previously mentioned substances.

7. A method for identifying a neurologically relevant gene, characterized in that nematodes, whose reaction to an exposure to at least one neurologically relevant material is known or has been established, are subjected to a mutagenesis, and in that the nematodes are, after that, exposed to at least one neurologically relevant material and the neurologically relevant gene is identified by establishing a deviation from the known reaction.
